(19) United States
(12) Patent Application Publication
(10) Pub. No.: US 2002/0161314 A1
(43) Pub. Date: Oct. 31, 2002

(54) Arrangement for patient monitor

(76) Inventor: Malla Sarajarvi, Espoo (FI)

Correspondence Address: Daniel D Fetterley, Andrus Sceales Starke & Savall, Suite 1100, 100 East Wisconsin Avenue, Milwaukee, WI, 53202-4178, US

(21) Appl. No.: 09/959,089

(22) PCT Filed: Feb. 15, 2001
(86) PCT No.: PCT/FI01/00147

(30) Foreign Application Priority Data
Feb. 17, 2000 (FI) 20000356

(51) Int. Cl. A61B 5/00

(52) U.S. Cl. 600/573; 604/349

(57) ABSTRACT

The invention relates to an arrangement for a patient monitor arranged to measure one or more parameters from a patient from other than urine or the urinary tract. To improve the ergonomics of patient monitoring work, the arrangement comprises a sensor (8) that is arranged between a patient urinary tract catheter (6) and a urine collection container (7) or the like and that is arranged to measure at least the flow or volume of the patient's urine output, and means (9) for conveying the data measured to the patient monitor (2)

The invention relates to an arrangement for a patient monitor arranged to perform one or more parameters on a patient from other than urine or the urinary tract.

For example in anaesthetic and intensive care, it is vital to continuously monitor a patient's condition, wherefore a special patient monitor is used for monitoring the patient's condition, the monitor measuring different parameters from the patient, e.g. EKG, NIBP, oxygen saturation, respiratory gases and invasive pressures etc. The number of parameters to be measured may depend on for example the situation at hand etc.

In addition to the above measurements, particularly in anaesthetic and intensive care it is important to follow, not only the above parameters, but also kidney function. In monitoring kidney function, hourly diuresis is important, since it expresses not only the reserves of the circulatory organs, but also the sufficiency of fluid treatment. The information should be given as uptake-with-respect-to-time, i.e. ml/5 min, ml/15 min, ml/30 min and ml/h, including cumulative uptake.

In presently used arrangements, the parameters used in patient monitoring are monitored by means of a patient monitor. However, for example urine is presently measured completely manually, whereby a nurse ocularly estimates the patient's urine output ml/h from an hourly urine collection container. In practice, the arrangement is laborious, since hospital personnel spend a lot of time in determining the amount of hourly urine.

Instead of manual measurement, urine can be measured more technically by the use of a special urine measurement device, in which hourly urine circulates through the device and the device measures the patient's hourly urine. For example U.S. Pat. No. 5,891,051 discloses such a device. The device disclosed in U.S. Pat. No. 5,891,051 is a separate monitor that cannot be connected to a larger monitoring unit. The device can also be used to register a patient's core temperature by the use of a special catheter provided with a sensor.

In present arrangements, urine flows along a catheter to an hourly urine collection container having a volume of about 500 ml. Once an hour, the hospital personnel have to empty the urine collection container into a larger urine collection bag. At present, the information is either manually added to a patient data system or the information is written down in the patients medical records (anaesthesia or intensive care form). The drawbacks include e.g. human factors, i.e. erroneous notes, forgetting etc. Further problems are caused by the trouble related to manual processing of the material, e.g. input of data in the system etc. It should be noted that the different patient-related measures very often have to be taken in a very pressing and stressful situation, which in itself is prone to cause human errors.

The object of the invention is to provide an arrangement that eliminates known prior art drawbacks. This is achieved with the arrangement of the invention, which is characterized in that the arrangement comprises a sensor arranged between a patient urinary tract catheter and a urine collection container or the like and arranged to measure at least the flow or volume of the patient's urine output, and means for conveying the data measured to the patient monitor.

The advantage of the invention is particularly that urine measurement, which previously caused extra work, can be monitored as one parameter in addition to other parameters. A special object of the invention is to achieve transfer of a signal obtained from urine measurement without manual input in the same monitor that monitors one or more measuring parameters from other than urine or the urinary tract. A further object is to display the urine measuring result on the display of the monitor simultaneously with some other parameter. Thus, no separate devices are required for measuring urine. This is of essential importance in practice, since intensive care units and operating theatres nowadays comprise such a high number of separate medical devices and related tubes and cables as to stress the tolerance of the medical staff to the utmost. The solution of the invention also ergonomically improves the medical staffs work compared with prior art, since utilization of the invention allows patients to be monitored entirely by means of the patient monitor, which facilitates work, since all necessary information is available on the patient monitor.

In the following, the invention will be described in detail by means of embodiments shown in the accompanying drawing, in which

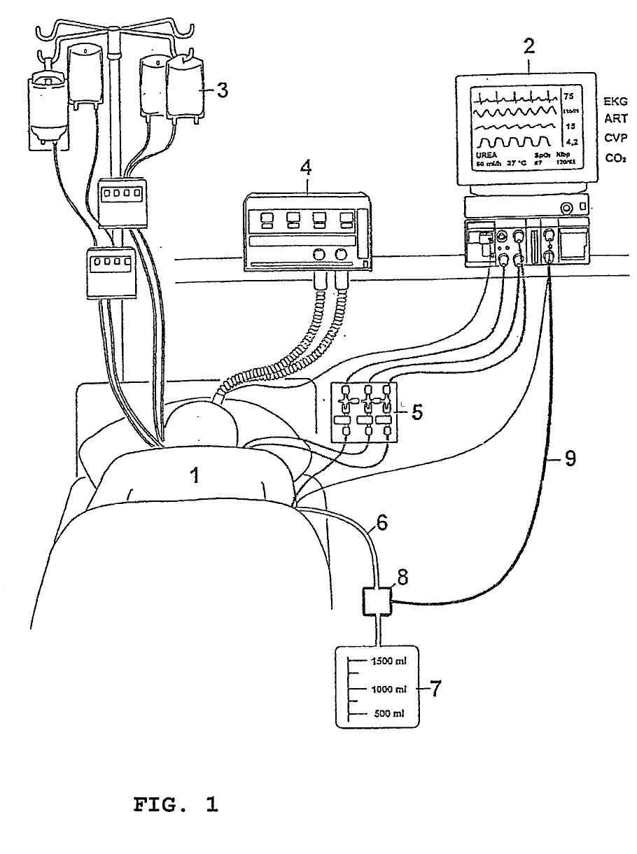

FIG. 1 schematically shows the arrangement of the invention in a patient care situation,

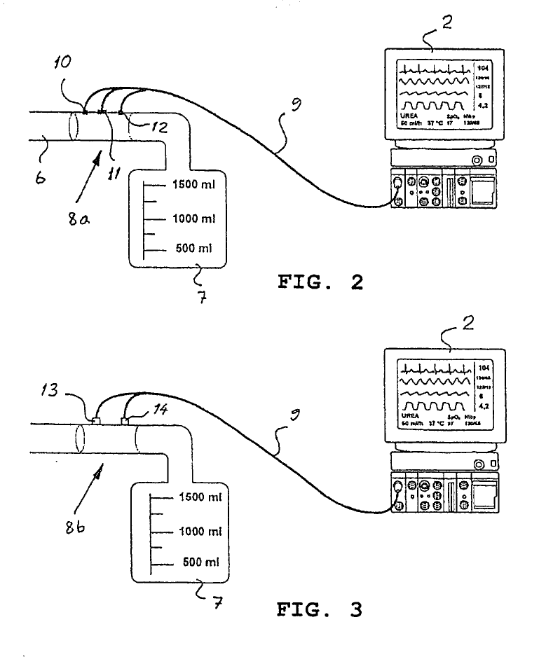

FIG. 2 schematically shows a first embodiment of the arrangement of the invention,

FIG. 3 schematically shows a second embodiment of the arrangement of the invention,

FIG. 4 schematically shows a third embodiment of the arrangement of the invention, and

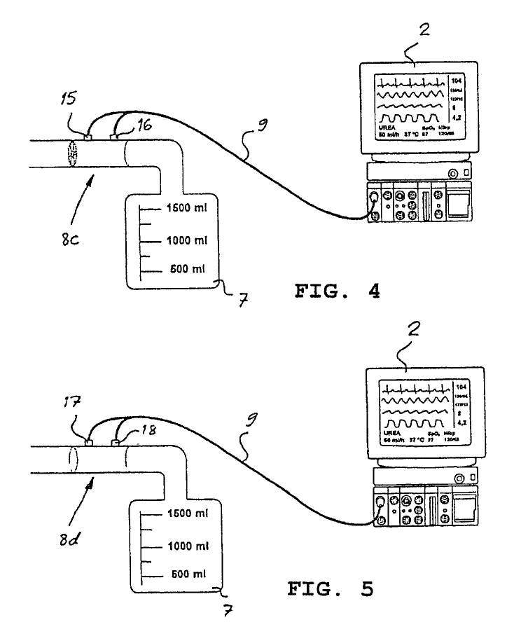

FIG. 5 schematically shows a fourth embodiment of the arrangement of the invention.

FIG. 1 schematically shows the arrangement of the invention in a patient care situation. In FIG. 1, reference 1 denotes a patient. In FIG. 1, reference 2 denotes a patient monitor arranged to measure different patient parameters, such as EKG, NIBP, oxygen saturation, respiratory gases, invasive pressures etc. The structure and use of a patient monitor are known per se to a person skilled in the art; hence, they are not explained in detail herein.

In FIG. 1, reference 3 denotes infusion bags connected to the patient. Reference 4 denotes a respirator and reference 5 an invasive pressure set. Said components are shown to indicate the number of tubes sometimes connected to a patient.

In FIG. 1, reference 6 denotes a urinary tract catheter, by means of which the patient's urine flows to a urine collection container 7. In accordance with the essential idea of the invention, the arrangement comprises a sensor 8 arranged between the patient urinary tract catheter 6 and the urine collection container 7 or the like. The sensor 8 is arranged to measure the flow or volume of the patient's 1 urine output. The sensor 8 can also be arranged to measure the momentary urine flow and/or the volume cumulated in a unit of time. Furthermore, the sensor can be arranged to measure urine temperature. The arrangement further comprises means for conveying the data measured to the patient monitor 2. The means 9 may be for example suitable conductors for transferring the data in electronic form to the patient monitor.

The essential advantage of the invention is that it enables routine urine measurement monitoring as one parameter in addition to other parameters without need for a separate device to measure this parameter. The arrangement of the invention substantially improves the ergonomics of work related to patient monitoring compared with the prior art. In practice, this is very important, since the number of different devices and the tubes and cables connected thereto is so high for example in operating theatres that it even causes risky situations etc., as was stated before. Separate devices, cables and tubes connected thereto sometimes also cause trouble when a patient is being moved. Such a situation arises easily for example when for some reason a patient has to be moved fast from one place to another, whereby a separate device may be forgot due to the hurry. Such forgetfulness may cause for example a loose device to fall from a support surface onto the floor, which always causes extra work and checks and, in the worst case, the device may be damaged and has to be replaced etc.

It is also essential to the invention that a conventional normal urinary tract catheter can be used as the urinary tract catheter; hence, no special catheter type is needed.

There are many ways to implement the sensor 8 arranged between the urinary tract catheter 6 and the urine collection container 7 or the like. FIG. 2 shows a first embodiment. In FIG. 2. the same references have the same significance as in FIG. 1. The exemplary embodiment shown in FIG. 2 is a flow sensor 8a based on heat transfer. Reference 10 denotes a temperature 1 measuring sensor, reference 11 a heater and reference 12 temperature 2 measurement.

FIG. 3 shows a second exemplary embodiment, which is a sensor 8b based on ultrasound. Reference 13 denotes input of a signal in a transmitter and reference 14 output of the signal from a receiver.

FIG. 4 shows an exemplary embodiment using a sensor 8c based on a turbine. Reference 15 shows input of power to the sensor measuring the speed of rotation of the turbine and reference 16 an output signal from the sensor.

FIG. 5 shows an exemplary embodiment using a sensor 8d based on a pressure difference. The pressure difference caused by a flow resistance installed inside a tube is measured between the points denoted by references 17 and 18.

The operation and structure of the sensors described above are known per se to a person skilled in the art; hence, they are not described in detail herein. In the embodiments of FIGS. 3 to 5, the sensor used to measure temperature can be placed for example as shown in FIG. 2; e.g. sensor 10, at a suitable point so that urine temperature and flow data are measured at substantially the same point.

The urine temperature measured by means of sensor 8, 8a, 8b, 8c, 8d can also be used to calculate core temperature, which is important for example in drowning cases and burns. It is to be noted that the invention also allows the core temperature to be obtained without a special catheter provided with a sensor.

The above exemplary embodiments are in no way intended to restrict the invention, but the invention can be freely modified within the scope of the claims. Accordingly, it is apparent that the arrangement of the invention and its details do not necessarily have to be as described above, but other solutions are also feasible. After measurement, instead of a urine collection container, the patient's urine output can be conveyed to another container or alternatively poured down the drain if no other measurements are performed on the urine. The invention also allows measurement data to be obtained as a function of time, which was not successful previously when urine was conventionally measured for example manually.

1. An arrangement for a patient monitor (2) arranged to measure one or more parameters from a patient (1) from other than urine or the urinary tract, characterized in that the arrangement comprises a sensor (8, 8a, 8b, 8c, 8d) arranged between a patient urinary tract catheter (6) and a 5 urine collection container (7) or the like and arranged to measure at least the flow or volume of the patient's urine output, and means (9) for conveying the data measured to the patient monitor (2).

2. An arrangement as claimed in claim 1, characterized in that the sensor (8, 8a, 8b, 8c, 8d) is arranged to measure also urine temperature.

3. An arrangement as claimed in claim 1 or 2, characterized in that the sensor comprises a flow sensor (8a) based on heat transfer.

4. An arrangement as claimed in claim 1 or 2, characterized in that the sensor comprises a flow sensor (8b) based on ultrasound.

5. An arrangement as claimed in claim 1 or 2, characterized in that the sensor comprises a flow sensor (8c) based on a turbine.

6. An arrangement as claimed in claim 1 or 2, characterized in that the sensor comprises a flow sensor (8d) based on a pressure difference.

7. An arrangement as claimed in claim 2, characterized in that the arrangement is arranged to calculate the patients core temperature by means of the information obtained from the urine temperature measurement.

8. An arrangement as claimed in any one of claims 1 to 7, characterized in that the sensor (8) is arranged to measure the momentary urine flow and/or the volume cumulated in a unit of time.

9. An arrangement as claimed in any one of claims 1 to 8, characterized in that the patient monitor (2) is arranged to measure the patient's EKG, NIBP, oxygen saturation and respiratory gases.
